(19) United States
(12) Patent Application Publication
(10) Pub. No.: US 2009/0220529 A1
(43) Pub. Date: Sep. 3, 2009

(54) Anticancer Drugs Conjugated to Antibody via an Enzyme Cleavable Linker

(75) Inventors: Andre Trouet, Herent (BE); Vincent Dubois, Gif sur Yvette (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Diatos, S.A., Paris (FR)

(21) Appl. No.: 12/282,441

(22) PCT Filed: Mar. 10, 2006
(86) PCT No.: PCT/IB2006/001185

§ 371 Date: Jan. 12, 2009

(51) Int. Cl. A61K 39/00; C07K 17/00

(52) U.S. Cl. 424/179.1; 530/391.1

(57) ABSTRACT

This invention relates to the field of antibody-drug conjugates, and more particularly antibody-drug conjugates that are intended for the treatment and/or diagnosis of diseases such as tumors and/or inflammatory reactions.

### BACKGROUND OF THE INVENTION

This invention relates to the field of antibody-drug conjugates, and more particularly antibody-drug conjugates that are intended for the treatment and/or diagnosis of diseases such as tumors and/or inflammatory reactions.

International publication numbers WO 96/05863 and WO 00/33888 describe tumor activated prodrugs, capable of being converted to active therapeutic compounds (preferably anticancer drug) in vivo by certain chemical and enzymatic modifications of their structure. These prodrugs are antitumoral drugs inactivated by linking to oligopeptides, which, due to their size, prevent the drug from entering all cells by diffusion or transport. Such oligopeptide should remain stable in the bloodstream but be sensitive to enzymes, released extracellularly by cancer cells (e.g., neprilysin), allowing the prodrug to be cleaved into the active drug in the immediate surroundings of the tumor while remaining intact and inactive in the surroundings of most non-tumor tissues. However, the selective activation of these prodrugs is not dependent on antigens that can be modulated by the tumor cells and is directly related to essential malignant characteristics of tumors namely their invasiveness and induction of neoangiogenesis.

Monoclonal antibody-therapeutic agent conjugates are another potential class of anticancer agents (Safavy et al., In Drug Targeting in Cancer Therapy, M. Page, ea., 257-275, 2002; Stan et al., Cancer Res, 59:115-121, 1999; Florent et al., J MedChem, 41: 3572-3581, 1998). A major goal has been the search for antibodies or peptides that can be specifically internalized by tumor cells upon binding to overexpressed cell-surface antigens, receptors or ligands (Nielsen et al., Pharm. Sci. Technol. Today, 3: 282-291, 2000; Trail et al., Cancer Immunol Immunother, 52: 328-337, 2003; Gao et al., J Immunol Methods, 274: 185-197, 2003; Gao et al., Bioorg Med Chem, 10: 4057-4065, 2002).

The task is however not as easy as it appears and a number of technical difficulties had first to be solved:

a) the requirement to have a bond between the therapeutic agent and the antibody that remains stable in the blood and extracellular spaces. This is indeed of importance in order not to have a leakage of the therapeutic agent before the interaction of the antibodies with their targets. Therefore a stable covalent bond is a first necessity;

b) given the stability of the therapeutic agent to carrier linkage, it is necessary for the therapeutic agent to be released from its antibody after fixation on the target cell and its internalization into the cell. Since antibodies can only interact with cell surface antigens it becomes obvious that the subsequent endocytosis of the antibodies by the target cells could provide an activation mechanism. Indeed after endocytosis the antibody-therapeutic agent conjugates reaches the lysosomes and the acidic pH prevailing in these organelles and their hydrolase content could insure a release of the therapeutic agent from its carrier. This requires that the bond between the therapeutic agent and the antibody is sensitive to one or more hydrolases or to the acidic pH and that the therapeutic agent is released in an active form able to diffuse into the cytosol and/or the nucleus to exert its effect;

c) in order to achieve this activation it is required that the antibody is endocytosed upon its interaction with the surface antigen. Only a fraction of the monoclonal antibodies behave in this way;

d) generally, there is a low number of tumor-related antigens at the cell surface, and

e) the number of therapeutic agents that can be linked to an antibody is relatively low (between 5 to 10) in order not to interfere with its antigen binding properties. Therefore it is important to target and to link very potent therapeutic agents.

These technical constraints have mostly been overcome. One demonstration of the clinical potential for such a strategy invoked the cell internalizing anti-CD33 antibody P67.6 conjugated to calicheamicin for use against acute myeloid leukemia that has resulted in the FDA approved drug Mylotarg™ (Hamann I, Bioconjug Chem, 13: 40-46, 2002), but nevertheless no clinically successful monoclonal-therapeutic agent conjugate has been developed yet for the treatment of solid tumors.

The problems still facing the use of monoclonal antibodies as anticancer therapeutic agent carriers can be listed as follows: 1) there are very few, if not at all, true tumor specific antigens accessible at the cell surface; 2) tumors can escape the action of the antibody-therapeutic agent conjugates by the presence or the selection of tumor cells devoid of the specific antigens. The antibody-therapeutic agent conjugates have very little, if any bystander effects. This is due to the intracellular activation of the targeted therapeutic agent and, in most cases, the very high affinity of the therapeutic agent for its intracellular sites of action. As a result the non-antigenic cells are very likely to escape the effects of the therapeutic agent released inside the surrounding antigen-bearing cells; 3) only a very small percentage of monoclonal antibodies reach the tumor and a great amount may accumulate in normal tissues such as the liver and the reticulo-endothelial system to become activated intracellularly and exert toxic effects.

Therefore, in spite of the advances in the art, there continues to be a need for the development of improved therapeutic agents, for example for the treatment of neoplastic diseases e.g., cancer, tumors or inflammatory diseases in mammals (e.g., human).

### BRIEF SUMMARY OF THE INVENTION

The present invention relates to a novel compound of use in the improved delivery of therapeutic agents into target cells or tissues, composition comprising the same and uses thereof.

The compounds are more specifically new antibody-therapeutic agent conjugates, wherein the antibody is a non-internalizing antibody and the therapeutic agent is covalently linked to the antibody via an oligopeptide arm, which provide numerous benefits, including high specificity of action, and reduced toxicity compared to the therapeutic agent itself. The invention is generally related to methods for treating a disease with an antibody-therapeutic agent conjugate, methods of synthesizing an antibody-therapeutic agent conjugate, and compounds that are useful as antibody-therapeutic agent conjugate, or useful in the synthesis of these molecules.

Therefore, benefits of the present invention can be obtained using antibodies to deliver therapeutic agents (e.g., chemotherapeutic agents) more selectively to target cells, typically via recognition of epitopes overexpressed in target tissues and activation by enzymes released extracellularly specifically by cells of the target tissue.

### DETAILED DESCRIPTION OF PREFERRED EMBODIMENTS

This invention is directed to a compound comprising a non-internalizing antibody linked to a therapeutic agent via an oligopeptide and its use to deliver therapeutic agents (e.g., chemotherapeutic agents) more selectively to target cells (e.g., tumor cells), typically via recognition of an epitope overexpressed in the target tissue and by the extracellular specific cleavage of the oligopeptide between the therapeutic agent and the antibody.

The therapeutic agent is linked directly or indirectly to an oligopeptide, which in turn, is linked directly or indirectly to a non-internalizing antibody. A linker group between the therapeutic agent and the oligopeptide may optionally be present. A spacer group between the therapeutic agent and the non-internalizing antibody may optionally be, present. The oligopeptide is at least 3 amino acids long and preferably 3 to 8 amino acids long. Preferably the compound of the invention is characterized by the susceptibility of the oligopeptide to cleavage by enzymes released extracellularly by cells of the target tissue (e.g., tumor cells).

More particularly, the compound of the invention is a modified form of a therapeutic agent and comprises several portions, including:

(1) a therapeutic agent or marker,

(2) an oligopeptide cleavable selectively by at least one enzyme that is present only or preferably close to or at said target cells,

(3) a non-internalizing antibody,

(4) optionally, a linker group, and

(5) optionally, a spacer group.

The non-internalizing antibody is directly or indirectly linked to the cleavable oligopeptide at a first attachment site of the cleavable oligopeptide. The cleavable oligopeptide is directly or indirectly linked to the therapeutic agent at a second attachment site of said cleavable oligopeptide. If the non-internalizing antibody and the cleavable oligopeptide are indirectly linked, then a spacer group is present. If the cleavable oligopeptide and the therapeutic agent are indirectly linked, then a linker group is present. Direct linkage of two portions of the compound of the invention means a covalent bond exists between the two portions. The non-internalizing antibody and the cleavable oligopeptide are therefore directly linked via a covalent chemical bond at the first attachment site of the cleavable oligopeptide, e.g. the N-terminus of the cleavable oligopeptide. When the cleavable oligopeptide and the therapeutic agent are directly linked then they are covalently bound to one another at the second attachment site of the cleavable oligopeptide. The second attachment site of the cleavable oligopeptide is typically the C-terminus of the cleavable oligopeptide, but may be elsewhere on the cleavable oligopeptide. Indirect linkage of two portions of the compound means each of the two portions is covalently bound to a linking moiety. In an alternative embodiment, the compound of the invention has indirect linkage of the non-internalizing antibody and the cleavable oligopeptide. Therefore, the non-internalizing antibody is covalently bound to the spacer group which, in turn, is covalently bound to the cleavable oligopeptide. In another alternative embodiment, the compound of the invention has indirect linkage of the cleavable oligopeptide to the therapeutic agent. Therefore, the cleavable oligopeptide is covalently bound to the linker group which, in turn, is covalently bound to the therapeutic agent. In another alternative embodiment, the compound of the invention has indirect linkage of the non-internalizing antibody and the cleavable oligopeptide and indirect linkage of the cleavable oligopeptide to the therapeutic agent.

In another aspect of the invention, the orientation of the compound may be reversed so that a non-internalizing antibody is directly or indirectly linked to the cleavable oligopeptide at the C-terminus of the cleavable oligopeptide and the therapeutic agent is directly or indirectly linked to the N-terminus of the cleavable oligopeptide. Thus, in the alternative embodiment, the first attachment site of the cleavable oligopeptide may be the C-terminus of the cleavable oligopeptide and the second attachment site by the cleavable oligopeptide may be the N-terminus of the cleavable oligopeptide. The spacer group may be optionally present between the non-internalizing antibody and the cleavable oligopeptide. The linker group may optimally be present between the therapeutic agent and the cleavable oligopeptide. The alternative embodiment of the compound of the invention functions in the same manner as does the primary embodiment.

Indirect linkage will occur through a “spacer” moiety between the non-internalizing antibody and the cleavable oligopeptide and through a “linker” moiety between the cleavable oligopeptide and the therapeutic agent. Suitable spacer and linker include bi and multifunctional organic radicals independently selected from substituted or unsubstituted alkyl, substituted or unsubstituted heteroalkyl, substituted or unsubstituted aryl, substituted or unsubstituted heteroaryl, aldehydes, acids, esters, ethers, thioethers, anhydrides, sulphydryl or carboxyl groups, such as maleinido derivatives, maleimido cyclohexane derivatives, maleimido benzoic acid derivatives, maleimidocaproic acid derivatives and succinimido derivatives or may be derived from cyanogen bromide or chloride, succinimidyl esters or sulphonic halides and the like.

The typical orientation of these portions of the compound is as follows: (non-internalizing antibody)-(optional spacer group)-(cleavable oligopeptide)-(optional linker group)-(therapeutic agent or marker).

The compound of the invention is typically cleavable within its cleavable oligopeptide portion.

In a first embodiment, for the compound of the invention to be effective, the compound typically undergoes in vivo modification producing the therapeutic agent or an active derivative of the therapeutic agent that is able to enter the target cell. A first cleavage within the cleavable oligopeptide portion of the compound may leave a fragment of the compound that is competent for transport into the target cell, as one of the cleavage products. Alternatively, further cleavage by one or more peptidases may be required to result in a transport-competent fragment of the compound. The active or transport-competent fragment of the compound has at least the therapeutic agent and is that part of the compound that can enter the target cell to exert a therapeutic effect directly or upon further conversion within the target cell.

The structures of the non-internalizing antibody and cleavable oligopeptide are selected to limit clearance and metabolism of the compound by enzymes that may be present in blood or non-target tissues and are further selected to limit transport of the compound into cells. The non-internalizing antibody blocks cleavage of the compound by peptidases in vivo in the blood (or the circulatory structure) or non-target tissue extracellular space and, additionally, may act in providing preferable charge or other physical characteristics to the compound.

The amino acid sequence of the cleavable oligopeptide is selected for susceptibility to cleavage by at least one enzyme preferably released by target cells or cells in the environment of the target cells. Compounds having cleavable oligopeptides of varying length may be used in the invention, but cleavable oligopeptides of at least 3 amino acids are preferred and cleavable oligopeptides of 3 to 8 amino acids are especially preferred. Preferably, the target cells are tumor cells, stromal cells, endothelial cells of tumors or cells, such as macrophages, neutrophils, and monocytes, participating in inflammatory reactions, especially those associated with rheumatic diseases.

Preferred enzymes associated with target cells are CD10 (CALLA or neprilysin) and TOP (Thimet oligopeptidase).

Advantageously, it is desirable to make a therapeutic agent, especially an antitumor and/or anti-inflammatory therapeutic agent, inactive by modification of the therapeutic agent to the compound form. Therefore, modification of the therapeutic agent to a compound can also reduce some of the side effects of the therapeutic agents.

The compound of the invention is administered to the patient, carried through the blood stream in a stable form, and when in the vicinity of a target cell, binds to an antigen and is modified by at least one target tissue-associated enzyme (e.g., peptidase). Since the enzyme activity is only minimally present within the extracellular vicinity of normal cells, the cleavable oligopeptide is not generally or is poorly cleaved outside target tissues and its transport-competent fragment (including the therapeutic agent) gains the normal cells only minimally at best.

In the vicinity of target cells (e.g., tumor), when the non-internalizing antibody binds its antigen, the increased presence of the relevant enzyme in the local environment causes cleavage of the cleavable oligopeptide.

In a second embodiment, for the compound of the invention to be effective, the compound typically undergoes in vivo modification producing a portion of the therapeutic agent that is an extracellularly active biological entity (i.e., an entity that can exert its biological activity without having to enter a cell, e.g., tumor necrosis factors). The second embodiment of the compound of the invention functions in the same manner as does the first embodiment, except the therapeutic agent does not enter the target cell.

Advantageously, the combination of the targeting properties of non-internalizing antibodies with the extracellular activating process by target tissue associated enzymes (e.g., tumor-selective peptidases), allows the therapeutic use of non-internalizing antibodies to deliver a therapeutic agent load selectively within the target cell environment (e.g., target tissue). Consequently, the therapeutic agent activation process of the antibody-therapeutic agent compound of the invention is independent of the endocytic and lysosomal uptake.

The compounds of the present invention have clearly major advantages:
the choice of tumor selective antibodies is not restricted by the requirement of internalizing antibodies;
the relative selectivity of antibodies is increased and completed by that of the choice of the cleavable oligopeptide and vice-versa;
the antibody-therapeutic agent compounds of the invention have bystander effects;
in certain embodiments, the non-internalizing antibodies can be directed not only to the tumor cells themselves but also to the endothelial and stromal cells as well as the extracellular matrix components of the tumor environment.

The invention therefore first has as its object an antibody-therapeutic agent compound comprising:

(1) a therapeutic agent or marker,

(2) an oligopeptide that can be cleaved selectively by a target tissue-associated enzyme that is present only or preferably close to or at said target cells (“cleavable oligopeptide”)

(3) a non-internalizing antibody, which specifically binds to an antigen of the target tissue,

(4) optionally, a linker group, which separates the cleavable oligopeptide from the therapeutic agent, and

(5) optionally, a spacer group, which separates the non-internalizing antibody from the cleavable oligopeptide so as to make possible or to facilitate the cleavage of the cleavable oligopeptide,

wherein the cleavable oligopeptide is directly linked to the non-internalizing antibody, or indirectly through the spacer group, at a first attachment site of the cleavable oligopeptide and the cleavable oligopeptide is directly linked to the therapeutic agent or indirectly linked through the linker group to the therapeutic agent at a second attachment site of the cleavable oligopeptide.

The terms “oligopeptide”, “peptide”, “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. These terms also encompass the term “antibody”.

The term “amino acid” refers to naturally occurring and non-natural amino acids, as well as amino acid analogs and amino acid mimetics that function in a manner similar to the naturally occurring amino acids. Naturally occurring amino acids are those encoded by the genetic code, as well as those amino acids that are non encoded by the genetic code or later modified, e.g., beta-alanine, D-serine, hydroxyproline, γ-carboxyglutamate, and O-phosphoserine. Amino acid analogs refers to compounds that have the same basic chemical structure as a naturally occurring amino acid i.e., a carbon that is bound to a hydrogen, a carboxyl group, an amino group, or an R group (see definition infra), e.g., homoserine, norleucine, methionine sulfoxide, methionine methyl sulfonium. Such analogs have modified R groups (e.g., norleucine) or modified backbones, but retain the same basic chemical structure as a naturally occurring amino acid, e.g. beta amino acids, amino acids in D conformation. Amino acid mimetics refers to chemical compounds that have a structure that is different from the general chemical structure of an amino acid, but that functions in a manner similar to a naturally occurring amino acid.

The referenced amino acids are represented in this invention either in three-letter code or in one-letter code, and it is submitted that three-letter and one letter amino acid code are well known to one skilled in the art.

The term “antibody” (Ab) refers to a protein comprising one or more peptidic chains encoded by immunoglobulin genes or fragments thereof that specifically binds and recognizes an epitope of an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The antibodies comprise IgG (including IgG₁, IgG₂, IgG₃, and IgG₄), IgA (including IgA₁ and IgA₂), IgD, IgE, or IgM, and IgY. As used herein, the term “antibody” is meant to include whole antibodies, including single-chain antibodies, and antigen-binding fragments thereof. Most preferably the antibodies are human antigen binding antibody fragments and include, but are not limited to, Fab, Fab′ and F(ab′)₂, Fd, single-chain Fvs (scFv), single-chain antibodies, disulfide-linked Fvs (sdFv), and fragments comprising either a V_{L} or V_{H} domain, and Nanobodies™ (see International publication number WO 94/04678).

The antibodies can be from any animal origin including birds and mammals. Preferably, the antibodies are human, murine, rabbit, goat, guinea pig, camelidae (e.g., camel, llamas) horse, or chicken.

The present invention further includes monoclonal, immunoadsorbed polyclonal, chimeric, humanized, intact antibody or isolated antibody.

The antibodies can be monospecific, bispecific, trispecific or of greater multispecificity.

An “intact antibody” comprises at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR^{X} or V_{L}) and a light chain constant region. The light chain constant region is comprised of one domain, C_{L}. The V_{H} and V_{L} regions can be further subdivided into regions of hypervariability, termed complementarily determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. Examples of binding include (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarily determining region (CDR).

An “isolated antibody” is one that has been identified and separated and recovered from a component of its natural environment. In preferred embodiments, the antibody will be purified to greater than 95% by weight of antibody as determined by the Lowry method, and most preferably more than 99% by weight.

Ordinarily, however, an isolated antibody can be prepared by at least one purification step.

“Single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; and Huston et al., Proc. Natl. Acad. Sci. USA, 85: 5879-5883, 1988). Such single chain antibodies are included by reference to the term “antibody fragment(s)”, and can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in International publication number Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins can be produced. See, Cabilly, U.S. Pat. No. 4,816,567 incorporated herein by reference in its entirety and for all purposes; and Queen et al., Proc. Natl. Acad. Sci. USA 86: 10029-10033, 1989.

“Monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology. “Polyclonal antibody” refers to a preparation of more than one (two or more) different antibodies to an antigen.

Such a preparation includes antibodies binding to a range of different epitopes.

“Chimeric antibodies” are those in which the Fc constant region of a monoclonal antibody from one species (typically a mouse) is replaced, using recombinant DNA techniques, with an Fc region from an antibody of another species (typically a human). For example, a cDNA encoding a murine monoclonal antibody is digested with a restriction enzyme selected specifically to remove the sequence encoding the Fc constant region, and the equivalent portion of a cDNA encoding a human Fc constant region is substituted. A CDR-grafted antibody is an antibody in which at least one CDR of a so-called “acceptor” antibody is replaced by a CDR “graft” from a so-called “donor” antibody possessing a desirable antigen specificity. Generally the donor and acceptor antibodies are monoclonal antibodies from different species; typically the acceptor antibody is a human antibody (to minimize its antigenicity in a human), in which case the resulting CDR-grafted antibody is termed a “humanized” antibody.

The graft may be of a single CDR (or even a portion of a single CDR) within a single V_{H} or V_{L} of the acceptor antibody, or can be of multiple CDRs (or portions thereof) within one or both of the V_{H} and V_{L}.

Methods for generating CDR— grafted and humanized antibodies are taught by Queen et al. U.S. Pat. No. 5,585,089, U.S. Pat. No. 5,693,761 and U.S. Pat. No. 5,693,762; and Winter U.S. Pat. No. 5,225,539, the contents of all of which are hereby incorporated by reference.

“Epitope” refers to groupings of molecules such as amino acid residues or sugar side chains at the surface of antigens that usually have specific three dimensional structural characteristics, as well as specific charge characteristics, and that are capable of specific binding by a monoclonal antibody.

The antibodies can also be described or specified in terms of their binding affinity. Preferred binding affinities include those with a dissociation constant or K_{d} less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Also included in the invention are modified antibodies to cell surface antigen.

“Modified antibody” refers to antibodies, such as monoclonal antibodies, chimeric antibodies, and humanized antibodies, which have been modified by, e.g., deleting, adding, or substituting portions of the antibody. For example, an antibody can be modified by deleting the constant region and replacing it with a constant region meant to increase half-life, e.g., serum half-life, stability or affinity of the antibody.

It can be desirable to couple more than one therapeutic agent or marker moiety to an antibody of the invention. By poly-derivatizing the antibodies of the invention, several therapeutic agent or marker strategies can be simultaneously implemented, an antibody can be made useful as a contrasting agent for several visualization techniques, or a therapeutic antibody can be labeled for tracking by a visualization technique. In one embodiment, multiple molecules of a therapeutic agent or marker moiety are coupled to one antibody molecule. In another embodiment, more than one type of moiety can be coupled to one antibody. Regardless of the particular embodiment, compounds of the invention with more than one moiety can be prepared in a variety of ways. For example, more than one moiety can be coupled to an antibody molecule via the cleavable oligopeptide of the invention, or linkers that provide multiple sites for attachment (e.g., dendrimers) can be used.

Advantageously, the antibody can increase in vivo the half-life time of the therapeutic agent linked to the cleavable oligopeptide in the circulatory structure. This result is attained in particular when the antibody reduces the renal elimination of the therapeutic agent or of therapeutic agent linked to the cleavable oligopeptide, whereby this elimination is based on the ultrafiltration through the kidneys of the compounds based on size and/or when the degradation by the hepatic metabolism of the compounds according to the invention is reduced. “To increase the half-life time” means to increase the mean residence time of the compound of the invention in the blood or to reduce the blood or plasmatic clearance compared to the therapeutic agent or marker themselves.

“Circulatory structure” is defined as body fluids, more particularly blood. In a specific embodiment the “circulatory structure” is those of a mammal, including tissues of the circulatory system.

The compounds of the invention are preferably stable in the circulatory structure. “Stable in the circulatory structure” means that less than about 20%, preferably less than about 2%, of the antibody-therapeutic agent conjugate of the invention is degraded or cleaved in the circulating blood (in particular by enzymes), during its preservation in human blood (at about 37 C for more than about 2 hours), is non-toxic for healthy cells, non-coagulating (i.e. prevents pro-coagulating properties of the therapeutic agent linked to the cleavable oligopeptide) or masking (i.e., preventing the therapeutic agent from acting on a cell until the latter has been released from the compound).

Advantageously, antibodies can also have one or more of the following properties: (i) prevent the non-specific cleavage and/or the degradation of the cleavable oligopeptide (e.g., hinders cleavage of the compound by enzymes present in whole blood); (ii) inhibit the biological effects of the therapeutic agent until the therapeutic agent has been released from the compound of the invention; (iii) increase the stability of the compound of the invention in the circulatory structure; (iv) increase the solubility in water, blood and/or the serum of the compound of the invention.

In a preferred embodiment, the size of the antibody can be between about 4,000 and about 800,000 Da, preferably between about 10,000 and about 150,000 Da.

A “non-internalizing antibody” has the property of reacting in physiological conditions (at 37° C. and pH 7) in vivo or in vitro, with cell surface antigens without being internalized in the cell bearing the antigens by a process of receptor/antigen mediated endocytosis.

Advantageously, the non internalizing property of the antibody results in the following properties that can be independently followed and easily characterized experimentally by one skilled in art:

1) a non-internalizing antibody remains at the cell surface for a prolonged time (e.g., between at least about 5 to about 30 minutes) until it is released from its antigens as a function of their association and dissociation constants (i.e., binding affinity). It can also be released if its corresponding antigen is shedded from the cell plasma membrane; or

2) the level of capture (“capture” is defined as the amount of antibodies associated with the cells at a given moment plus the amount of antibodies degraded intracellularly and released extracellularly as labelled fragments) of a non-internalizing antibody by the cells reaches a steady level after few minutes (between about 5 to about 30 minutes) that is not significantly different from that observed during an incubation at 4° C. and 37° C.; or

3) a non-internalizing antibody is not internalized into the cells by a receptor/antigen type of endocytosis and therefore does not reach the lysosomal system and is not degraded to a significant extent by lysosomal enzymes; or

4) a non-internalizing antibody can however be taken up intracellularly by fluid phase endocytosis and this capture depends linearly on their extracellular concentration and this can explain a slightly significantly higher capture at 37° C. compared to 4° C. as well as a small amount of intracellular degradation of said antibody.

The non-internalizing characteristic of an antibody can be best determined experimentally by evaluating the aforementioned characteristics of the non-internalizing antibody.

By way of example, a peculiar class of non-internalizing antibodies are those that interact with antigens and epitopes present at the surface of target tissue extracellular constituents such as those of the extracellular matrix.

Methods allowing to characterize or select non-internalizing antibodies are described in further detail below:

The main method involves an in vitro incubation (at 37° C. in an appropriate cell culture medium known from one skilled in art or otherwise indicated) of the non-internalizing antibodies with cells displaying the corresponding antigens at their cell surface. The antibodies are labelled either with a fluorescent marker or detected by labelled anti-antibody antibodies for microscopic observation (Immunofluorescence: Starling, et al., Cancer Research 51:2195-2972 (1991); Supra et al. Cancer Research 62, 7190-7194 (2002)), or are labelled with a radioactive marker for quantitative investigations. As a control the same test is performed with a non-imiunocompetent analog of the selected non-internalizing antibody with similar physico-chemical characteristics (molecular weight, isoelectric point) and biological properties (e.g., same Ig class, IgG type, IgG fragments such as Fab′₂, Fab, light or heavy chains, single chain antibodies such as nanobodies, etc.) Non-internalizing antibodies can be defined by one or several of the following criteria:

1° Immunofluorescent images indicate a surface and peripheral distribution of the non-internalizing antibodies with no or very few intracellular labelled granules (i.e., less than about 20% intracellular fluorescence compared to pericellular/extracellular fluorescence). Internalizing antibodies show, by contrast, a great number of intracellular fluorescent granules (i.e., more than about 80% intracellular fluorescence compared to pericellular/extracellular fluorescence).

2° At saturation of the antigen-binding sites and after a few minutes (between about 5 to about 30 minutes), the cell capture of non-internalizing antibodies does not differ significantly following an incubation at 4° C. and 37° C.; or.

3° After reaching a plateau of accumulation, the cell associated non-internalizing antibodies can be released by incubation at a pH<7 (preferably between about 4 and about 5.5) dissociating the antigen complexes at the cell surface. The amount of releasable internalizing antibodies, at acid pH, is then significantly lower (at least 20% or more preferably at least 10%) because of their intracellular localization; or

4° Incubation of non-internalizing antibodies with cells gives rise to low amounts of antibody degradation fragments and products. These products can be found intracellularly and in the extracellular medium if they can leave cells by cell permeation or exocytosis from the lysosomal compartment. The low amounts (less than 10%) of degraded material found with non-internalizing antibodies should not exceed those observed with the non-immunocompetent antibodies.

Examples of non-internalizing antibodies comprise; (i) Anti-CEA antibodies; (ii) Anti-CD20 antibody (Sapra and Allen, Cancer Res. 2002, 62, 7190-7194); (iii) B72.3 antibody (anti TAG-72) (Cancer Res. 1991, 51, 2965-72; (iv) ScFV anti P4G7, VEGFR-2 (J. Immunol. Methods. 2004, 289, 37-45).

In another embodiment, a non-internalizing antibody is derived from the L6 murine monoclonal antibody disclosed in U.S. Pat. No. 4,906,562 and U.S. Pat. No. 4,935,495. L6 is a non-internalizing antibody active against a ganglioside antigen expressed by human carcinoma cells derived from human non-small cell lung, breast, colon or ovarian carcinomas. The hybridoma expressing L6 and identified as L6 was deposited under the terms of the Budapest Treaty on Dec. 6, 1984 at the ATCC and is available under the accession number HB 8677. The hydridoma is cultured and the desired antibody is isolated using the standard techniques referenced above. A chimeric form of the L6 antibody, if desired, is described in International publication number WO 88/03145.

Methods of generating antibodies or antibody fragments of the invention typically include immunizing a subject (a non-human subject such as a mouse or rabbit) with purified antigen or with a cell expressing antigen. Many techniques of generating antibodies or antibody fragments are standard in the art.

For preparation of monoclonal or polyclonal antibodies, any technique known in the art can be used (see, e.g., Kohler & Milstein, Nature 256: 495-497 (1975); Kozbor et al., Immunology Today 4: 72 (1983); Cole et al., pp. 77-96 in MONOCLONAL ANTIBODIES AND CANCER THERAPY, Alan R. Liss, Inc. (1985)).

In general, therapeutic agents can be conjugated to the cleavable oligopeptide of the invention, for example, by any suitable technique, with appropriate consideration of the need for pharmacokinetic stability and reduced overall toxicity to the patient. A therapeutic agent can be coupled to the oligopeptide portion either directly or indirectly (e.g., via a linker group). By way of example, when the oligopeptide is directly linked to the therapeutic agent, the covalent bond can then be produced at the N-terminal or C-terminal end of the oligopeptide according to its orientation, or at any other site of the oligopeptide (for example at the lateral chain of one of the amino acids). A direct reaction between an agent and an oligopeptide is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. Suitable linker group include bi- and multi-functional or divalent organic radicals independently selected from substituted or unsubstituted alkyl, substituted or unsubstituted heteroalkyl and substituted or unsubstituted aryl, aldehydes, acids, esters and anhydrides, sulphydryl or carboxyl groups, such as maleimido benzoic acid derivatives, maleimidocaproic acid derivatives and succinimido derivatives or may be derived from cyanogenc bromide or chloride, succinimidyl esters or sulphonic halides and the like. The functional groups on the linker moiety used to form covalent bonds between linker and therapeutic agent or marker on the one hand, as well as linker and oligopeptide moiety on the other hand, may be different types of functional groups, including amino, hydrazino, hydroxyl, thiol, maleimido, carbonyl, and carboxyl groups. The linker moiety may include a short sequence of from 1 to 8 amino acid residues that optionally includes a cysteine residue through which the linker moiety bonds to the oligopeptide. A linker group allows to distance the therapeutic agent from the oligopeptide in order to avoid interference with the interaction of the oligopeptide with target tissue specific peptidases. A linker group can also serve to increase the chemical reactivity of a substituent on a moiety or an oligopeptide, and thus increase the coupling efficiency. An increase in chemical reactivity can also facilitate the use of moieties, or functional groups on moieties, which otherwise would not be possible. Accordingly, when the oligopeptide is indirectly linked to the therapeutic agent, the linker group may have several functions such as to facilitate the cleavage between the oligopeptide and the therapeutic agent or marker, to provide a suitable chemical bonding means between the oligopeptide and the therapeutic agent or marker, to improve the synthesis process of the compound of the invention, to improve the physical properties of the substance of the therapeutic agent or marker, or to provide an additional mechanism of the intracellular or extracellular release of substance of the therapeutic agent or marker. Suitable linkage chemistries include maleimidyl linkers, alkyl halide linkers (which react with a sulfhydryl on the oligopeptide moiety) and succinimidyl linkers (which react with a primary amine on the oligopeptide moiety) (e.g., succinimidyl 4-(N-maleimidomethyl)cyclohexane-1-carboxylate). Several primary amine and sulfhydryl groups are present on oligopeptides, and additional groups can be designed into recombinant oligopeptide molecules. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues (see, e.g., U.S. Pat. No. 4,671,958). Cyanogen bromide or chloride derivative groups; carbonyldiimidazole, thiocarbonyldiimidazole of succinimide esters or sulfonic halides; phosgene, thiophosgene; or self-rearrangeable (or “self-immolative”) spacers (Schmidt et al., 2001) can also be used. Where a cytotoxic moiety is more potent when free from the compound or a portion of the compound of the present invention, it can be desirable to use a linker group which is cleavable during or upon penetration into a cell of the therapeutic agent linked to a portion of the cleavable oligopeptide, or which is gradually cleavable over time in the extracellular environment. A number of different cleavable linker groups have been described. The mechanisms for the intracellular release of a cytotoxic moiety agent from these linker groups include cleavage by reduction of a disulfide bond, by irradiation of a photolabile bond, by hydrolysis of derivatized amino acid side chains, and acid-catalyzed hydrolysis.

The “spacer group”, if present, according to the present invention covalently links the non-internalizing antibody to the cleavable oligopeptide. It is preferably hydrophilic and stable in the circulatory structure (e.g. the blood circulation or bloodstream).

A spacer is “stable in the circulatory structure” when less than about 20%, preferably less than about 10%, preferably even less than about 2%, of the spacer is degraded or cleaved (in particular by enzymes) in the circulating blood or during its preservation in human blood at about 37° C., for more than about 2 hours. Advantageously, by separating the antibody from the oligopeptide and by its preferably hydrophilic nature, the spacer group makes possible or facilitates the cleavage of the oligopeptide close to or at target cells. The spacer group can exhibit a length (or size) that can be on the order of the equivalent of about 1 to about 100 amino acids, preferably of about 1 to 20.

In general, the oligopeptides can be conjugated to the antibodies, for example, by any suitable technique, with appropriate consideration of the need for pharmacokinetic stability and reduced overall toxicity to the patient.

An oligopeptide can be coupled to a suitable antibody moiety either directly or indirectly (e.g., via a spacer group). A direct reaction between an oligopeptide and an antibody is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical spacer group can be used. The functional groups on the spacer group used to form covalent bonds between spacer and antibody on the one hand, as well as spacer and oligopeptide moiety on the other hand, may be different types of functional groups, including amino, hydrazino, hydroxyl, thiol, maleimido, carbonyl, and carboxyl groups. The spacer moiety may include a short sequence of from 1 to 4 amino acid residues that optionally includes a cysteine residue through which the spacer moiety bonds to the oligopeptide. Suitable spacer groups include bi- and multi-functional or divalent organic radicals independently selected from substituted or unsubstituted alkyl, substituted or unsubstituted heteroalkyl and substituted or unsubstituted aryl, aldehydes, acids, esters and anhydrides, sulphydryl or carboxyl groups, such as maleimido benzoic acid derivatives, maleimidocaproic acid derivatives and succinimido derivatives or may be derived from cyanogen bromide or chloride, succinimidyl esters or sulphonic halides and the like. A spacer group allows to distance the antibody from the oligopeptide in order to avoid interference with its binding capabilities and/or to facilitate the cleavage of the oligopeptide. A spacer group can also serve to increase the chemical reactivity of a substituent on the oligopeptide or an antibody, and thus increase the coupling efficiency. An increase in chemical reactivity can also facilitate the use of moieties, or functional groups on moieties, which otherwise would not be possible.

The spacer according to this invention consists of, or alternatively comprises, at least one group that is selected from among: the sequences of amino acids; the peptidomimetic agents; the pseudopeptides; the peptoids; the substituted alkyl, aryl or arylalkyl chains; the polyalkyl glycols; the polysaccharides; the polyols; the polycarboxylates; and the poly(hydro)esters. In another embodiment of the invention, the spacer thus can consist of, or alternatively comprise, a combination of at least two of these groups.

According to an advantageous embodiment of the invention, the spacer consists of or comprises about 1 to about 100, preferably about 1 to about 20, and very preferably about 2 to about 10, identical or different amino acids selected from the group that comprises the natural amino acids in conformation L or D, the genetically uncoded amino acids or the amino acids that cannot be recognized by an enzyme (e.g. the peptidases) that is present in the circulatory structure, such as the beta- or gamma-amino acids or the like. “Natural amino acids in conformation D” are defined as the amino acids that are normally coded by the genetic code but that instead of being naturally in conformation L are in conformation D. Generally, the genetically uncoded amino acids can be prepared by synthesis or can be derived from a natural source.

Preferred among the natural amino acids in conformation D are the hydrophilic amino acids that are selected from among: D-glutamine, D-asparagine, D-aspartic acid, D-glutamic acid, D-lysine, D-arginine, and D-histidine. Particularly preferred amino acids in conformation D are D-serine and D-threonine.

According to a preferred method of this invention, the spacer group comprises, or alternatively consists of, a sequence of identical amino acids that are selected from among: (l-serine)x, (d-serine)x, or (l-threonine)x (d-threonine)x, where x is an integer between about 1 and about 20, preferably between about 2 and about 15, and more preferably x=4, 8 or 12.

The term “alkyl”, by itself or as part of another substituent, means, unless otherwise stated, a straight or branched chain, or cyclic hydrocarbon radical, or combination thereof, which can be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl”, unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by CH₂CH₂CH₂CH₂, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “heteroalkyl”, by itself or in combination with another term, means, unless otherwise stated, a stable straight or branched chain, or cyclic hydrocarbon radical, or combinations thereof, consisting of the stated number of carbon atoms and at least one heteroatom selected from the group comprising O, N, Si and S, and wherein the nitrogen, carbon and sulfur atoms can optionally be oxidized and the nitrogen heteroatom can optionally be quaternized. The heteroatom(s) O, N and S and Si can be placed at any interior position of the heteroalkyl group or at the position at which the alkyl group is attached to the remainder of the molecule. Examples include, but are not limited to, CH₂CH₂OCH₃, CH₂CH₂NHCH₃, CH₂CH₂N(CH₃)CH₃, —CH₂SCH₂—CH₃, CH₂CH₂, S(O)CH₃, CH₂CH₂S(O)₂CH₃, CH—CHOCH₃, Si(CH₃)₃, —CH₂CH═NOCH₃, and CH═CHN(CH₃)CH₃. Up to two heteroatoms can be consecutive, such as, for example, CH₂NHOCH₃ and CH₂OSi(CH₃)₃. Similarly, the term “heteroalkylene” by itself or as part of another substituent means a divalent radical derived from heteroalkyl, as exemplified, but not limited by, CH₂CH₂SCH₂CH₂ and CH₂SCH₂CH₂NHCH₂. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives. Still further, for alkylene and heteroalkylene linking groups, no orientation of the linking group is implied by the direction in which the formula of the linking group is written.

The term “lower” in combination with the terms “alkyl” or “heteroalkyl” refers to a moiety having from 1 to 6 carbon atoms.

The terms “alkoxy”, “alkylamino” and “alkylthio” (or thioalkoxy) are used in their conventional sense, and refer to those alkyl groups attached to the remainder of the molecule via an oxygen atom, an amino group, or a sulfur atom, respectively.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively.

Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropy-ridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The heteroatoms and carbon atoms of the cyclic structures are optionally oxidized.

The terms “halo” or “halogen”, by themselves or as part of another substituent, mean, unless otherwise stated, a fluorine, chlorine, bromine, or iodine atom.

Additionally, terms such as “haloalkyl”, are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like. The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O and S. wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, and arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl”, “heteroalkyl”, “aryl” and “heteroaryl”) include both substituted and unsubstituted forms of the indicated radical. Preferred substituents for each type of radical are provided below.

Substituents for the allyl, and heteroalkyl radicals (including those groups often referred to as alkylene, alkenyl, heteroallkylene, heteroalkenyl, alkynyl, cycloalkyl, heterocycloalkyl, cycloallcenyl, and heterocycloalkenyl) are generally referred to as “alkyl substituents” and “heteroalkyl substituents”, respectively, and they can be one or more of a variety of groups selected from, but not limited to: OR′, ═O, ═NR′, ═NOR′, NR′R″, SR′, -halogen, SiR′R″R′″, OC(O)R′, C(O)R′, CO₂R′, —CONR′R″, OC(O)NR′R″, NR″C(O)R′, NR′—C(O)NR″R′″, NR″C(O)₂R′, NR—C(NR′R″R′″)═NR″″, NRC(NR′R″)═NR′″, S(O)R′, S(O)₂R′, S(O)₂NR′R″, NRSO₂R′, CN and NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl.

From the above discussion of substituents, one of skill in the art will understand that the term “alkyl” is meant to include groups including carbon atoms bound to groups other than hydrogen groups, such as haloalkyl (e.g., CF₃ and CH₂CF₃) and acyl (e.g., C(O)CH₂, C(O)CF₃, C(O)CH₂OCH₃, and the like).

Similar to the substituents described for the alkyl radical, the aryl substituents and heteroaryl substituents are generally referred to as “aryl substituents” and “heteroaryl substituents”, respectively and are varied and selected from, for example: halogen, OR′, ═O, ═NR′, ═NOR′, NR′R″, SR′, -halogen, SiR′R″R′″, OC(O)R′, C(O)R′, —CO₂R′, CONR′R″, OC(O)NR′R″, NR″C(O)R′, NR′C(O)NR″R′″, NR″C(O)₂R′, NR—C(NR′R″)═NR′″, S(O)R′, S(O)₂R′, S(O)₂NR′R″, NRSO₂R′, CN and NO₂, R′, N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

An important portion of the compounds of the invention is the cleavable oligopeptide. “Cleavable oligopeptide” means an oligopeptide that can be cleaved selectively by a target tissue-associated enzyme, preferably by an enzyme that is present only or preferably close to (e.g., in the environment of) or at the target cells (e.g., tumor cells). The “cleavable oligopeptide” allows for activation of the therapeutic agent or marker by cleaving the agent or marker from the remaining part of the compound.

“Target cells” are defined more particularly as the cells that are involved in pathology, that are preferred targets for therapeutic or diagnostic activity. These target cells are preferably one or more of the cells comprising, or alternatively consisting of, the cells of the following group: primary or secondary tumor cells (the metastases), stromal cells of primary or secondary tumors, neoangiogenic endothelial cells of tumors or tumor metastases, macrophages, monocytes, polymorphonuclear leukocytes and lymphocytes or polynuclear agents infiltrating the tumors and the tumor metastases.

“Target tissue” comprises target cells themselves (e.g., tumor cells) but also any cells in the environment of the target cells, such as the endothelial and stromal cells as well as the extracellular matrix components of the tumor environment.

“Selectively cleavable” or “cleaved selectively” is defined as cleavage dictated by the sequence to be cleaved. The sequence to be cleaved is preferably recognized by an enzyme that is present in the environment of the target cells, and it is degraded slightly or not at all in the circulatory structure or close to non-target cells. The expression “in the environment of the target cells” means that the enzyme is present either by itself or preferably close to or at the target cells. It should be noted that even if the cleavage is not carried out only close to or at the target cells, the fact that the cleavage is carried out preferably (or in large part or in the majority of the cases) close to or at the target cells makes this cleavage selective. The cleavage is called selective when the enzyme is in a larger concentration close to or at the target cells relative to the remainder of the organism.

“Target tissue-associated enzyme” is defined as a membrane enzyme or an enzyme that is released by the cells of the target tissue or the tissue in the near environment of the target tissue, or only by the target cells in the extracellular medium surrounding these target cells. In one embodiment of the invention, when the target tissue is a tumor, the enzyme can be selected from the group that comprises, or alternatively consists of, neprilysin (CD10), thimet oligopeptidase (TOP), prostate specific antigen (PSA), plasmin, legumain, collagenases, urokinase, cathepsins, and the matrix metallopeptidases (these enzymes being well known from one skilled in the art).

The enzyme comprises, or alternatively consists of, one or more enzymes selected from the group of peptidases, endopeptidases and lysosomal enzymes.

According to a preferred embodiment of this invention, the enzyme is a peptidase of the tumor cells, stromal cells of the tumors, neoangiogenic endothelial cells, macrophages or monocytes.

The selection of the amino acid sequence of the cleavable oligopeptide is based on the specific enzyme that is present in the environment of the target cells.

The cleavable oligopeptide preferably comprises, or alternatively consists of, between about 2 and about 10 amino acids, and preferably also about 3 to about 7 amino acids.

In some embodiments of the invention, the oligopeptide comprises, or alternatively consists of, one or more of the following amino acid sequences (preferably in conformation L): Arg-Leu, Arg-Phe, Arg-Val, Ala-Phe, Ala-Leu, Ala-Tyr, Cys-Arg, Cys-Asp, Cys-Phe, Gln-Phe, Gly-Asp, Gly-Phe, Gly-Leu, Gly-Gln, Gly-Gly, Gly-Pro, His-Ser, Ile-Ala, Leu-Gin, Leu-Gly, Leu-Leu, Leu-Phe, Leu-Tyr, Lys-Leu, Met-Leu, Pro-Phe, Pro-Tyr, Pro-Leu, Phe-Leu, Phe-Phe, Tyr-Ile, Tyr-Pro, Tyr-Leu, Val-Tyr, Val-Phe, Ser-Leu, and Ser-Lys.

In a preferred embodiment of the invention, the oligopeptide comprises, or alternatively consists of, one or more of the following sequences (preferably in conformation L): (Leu)y-(Ala-Leu)x-Ala-Leu or (Leu)y-(Ala-Leu)x-Ala-Phe wherein Leu is leucine, Ala is alanine, Phe is phenylalanine, y=0 or 1 and x=1, 2, or 3.

In another preferred embodiment of the invention, the oligopeptide comprises, or alternatively consists of, one or more of the following sequences (preferably in conformation L): Ala-Phe-Lys (SEQ ID No. 1), Ala-Leu-Ala-Leu (SEQ ID No. 2) or beta-Ala-Leu-Ala-Leu ((SEQ ID No. 3), Ala-Leu-Lys-Leu-Leu (SEQ ID No. 4), Ala-Tyr-Gly-Gly-Phe-Leu (SEQ ID No. 5), His-Ser-Ser-Lys-Leu-Gln-Leu (SEQ ID No. 6), Gly-Pro-Leu-Gly-Ile-Ala-Gly-Gln (SEQ ID No. 7), Cys-Asp-Cys-Arg-Gly-Asp-Cys-Phe-Cys (SEQ ID No. 8) and Ala-Leu-Lys-Leu-Leu (SEQ ID No 9).

One skilled in the art, however, knows other sequences of amino acids that can be cleaved selectively by a specific enzyme of tumor cells, such as those described in International publication number WO 00/33888 (see amino acid sequences described pages 13 to 15), WO 01/68145 (see amino acid sequences described SEQ ID Nos 1 to 210), WO 01/95943 (see amino acid sequences described pages 16 to 18), WO 02/00263 (see amino acid sequences described table 1, page 16), WO 02/100353 (see amino acid sequences described SEQ ID Nos 1 to 36), WO 02/07770 (see amino acid sequences described page 3) or WO 99/28345 (see amino acid sequences described SEQ ID Nos 1 to 125), each incorporated herein by reference in their entirety and for all purposes.

The enzymes according to the invention are able to selectively cleave the cleavable oligopeptide so as to make possible the release of the therapeutic agent or the release of the therapeutic agent linked to a portion of the cleavable oligopeptide. The expression “release of the therapeutic agent linked to a portion of the cleavable oligopeptide” is explained by the following example. If the cleavable oligopeptide is an amino acid sequence whose sequence is Ala-Leu-Ala-Leu, the therapeutic agent is doxorubicin (whereby the therapeutic agent directly linked to the cleavable oligopeptide is Ala-Leu-Ala-Leu-doxorubicin) and the enzyme is CD10 (neprilysin), then this enzyme cleaves the sequence of amino acids between Ala-Leu-Ala and Leu, thus releasing a Leu-doxorubicin product (i.e., a portion (or part) of the therapeutic agent) e.g. see International publication number WO 00/33888. This product is therefore defined as “therapeutic agent linked to a portion of the cleavable oligopeptide”.

“Extra-blood reactivation” or “reactivation in the extra-blood compartment” is defined as the cleavage of the oligopeptide of the compound of the invention by specific endopeptidases that are present in any organ or tissue (healthy or tumor, for example) other than the blood and preferably at target cells. The cleavage of oligopeptide (for example a peptide) results in the release of an active form of the therapeutic agent.

One skilled in the art can refer to Handbook of Proteolytic Enzymes (A. J. Barreft, N. D. Rawlings, and J. F. Woessner eds. Academic Press, 1998; incorporated herein by reference in their entirety and for all purposes) to characterize the substrate-specificity of enzymes.

“Therapeutic agent” is intended to mean a compound that, when present in a therapeutically effective amount, produces a desired therapeutic effect on a mammal, and whose action site is located or whose effect will be exerted on the surface or inside target cells. By way of example, a therapeutic agent of interest comprises, or alternatively consists of, an agent selected from the following group: a chemical agent, a polypeptide, a protein, a nucleic acid, an antibiotic, and a virus.

Said therapeutic agent is preferably an agent with anti-tumor, anti-angiogenic or anti-inflammatory therapeutic activity. The agent can have a target (for example a receptor) or extracellular or intracellular action site. It can also comprise a penetrating peptide sequence such as a sequence that is described in U.S. patent application Ser. No. 10/231,889. By way of example, the therapeutic agent comprises, or alternatively consists of, a agent selected from the following group of agents with anti-tumor therapeutic activity: vinca alkaloids such as vincristine, vinblastine, vindesine, vinorelbine; taxanes or taxoids such as paclitaxel, docetaxel, 10-deacetyltaxol, 7-epi-taxol, baccatin III, le xylosyltaxol; alkylating agents such as ifosfamide, melphalan, chloroaminophene, procarbazine, chlorambucil, thiophosphoramide, busulfan, dacarbazine (DTIC), mitomycins including mitomycin C, nitroso-ureas and derivatives thereof (for example, estramustine, BCNU, CCNU, fotemustine); platinum derivatives such as cisplatin and the like (for example, carboplatin, oxaliplatin); antimetabolites such as methotrexate, aminopterin, 5-fluorouracil, 6-mercaptopurine, raltitrexed, cytosine arabinoside (or cytarabine), adenosine arabinoside, gemcitabine, cladribine, pentostatin, fludarabine phosphate, hydroxyureas; inhibitors of topoisomerase I or II such as the camptothecin derivatives (for example, irinotecan and topotecan or 9-dimethylaminomethyl-hydroxy-camptothecin hydrochloride), epipodophyllotoxins (for example etoposide, teniposide), amsacrine; mitoxantrone; L-canavanine; antibiotic agents such as anthracyclines and, for example, adriamycin or doxorubicin, THP-adriamycin, daunorubicin, idarubicin, rubidazone, pirarubicin, zorubicin and aclarubicin, the analogs of anthracyclines and, for example, epiadriamycin (4′epi-adriamycin or epirubicin) and mitoxantrone, bleomycins, actinomycins including actinomycin D, streptozotocin, calicheamicin, duocarmycins, combretastatin; L-asparaginase; hormones; pure inhibitors of aromatase; androgens, analog-antagonists of LH-RH; cytokines such as interferon alpha (IFN-alpha), interferon gamma (IFN-gamma), interleukin 1 (IL-1), IL-2, IL-4, IL-6, IL-10, IL-12, IL-15, the tumor necrosis factor-alpha (TNF-alpha), the IGF-1 antagonists (insulin-like growth factor); the proteasome inhibitors; the farnesyl-transferase inhibitors (FTI); the epothilones; the maytansinoids; discodermolide; fostriecin; antibodies; the inhibitors of tyrosine kinases such as STI 571 (imatinib mesylate); endostatins; proteins, peptides, and anti-inflammatory cytokines. “Marker” is intended to mean a compound useful in the characterization of tumors or other medical condition, for example, diagnosis, progression of a tumor, and assay of the factors secreted by tumor cells. A Marker is further defined as enzymes, antibodies, fluorescent or phosphorescent chemical molecules, and molecules that can be used in scintigraphy. Examples of markers include, but are not limited to, coumarin, 7-amido-trifluoromethyl coumarin, paranitroanilide, 8-naphthylamide and 4-methoxy naphthylamide, fluorosceine, biotin, rhodamine, tetramethylrhodamine, GFP (green fluorescent protein), the agents that are used in scintigraphy as radioactive isotopes, and the derivatives of these compounds.

The term “cancer” refers to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous cell” or “cancer cell” is understood as a cell having specific structural properties, that can lack differentiation and be capable of invasion and metastasis. Examples of cancers are, breast, lung, brain, bone, liver, kidney, colon, and prostate cancer. (see DeVita, V. et al. (eds.), 2005, Cancer Principles and Practice of Oncology, 6th. Ed., Lippincott Williams & Wilkins, Philadelphia, Pa.; this reference is herein incorporated by reference in its entirety for all purposes).

In another embodiment, the invention encompasses pharmaceutically acceptable basic or acidic addition salts, hydrates, solvates, precursors, metabolites or stereoisomers of said compound of the invention.

The term “pharmaceutically acceptable salts” includes salts of the compounds of the invention, which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfaric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Another embodiment of the invention is also a composition that comprises, or alternatively consists of, as an active ingredient, at least one compound of the invention.

In still another embodiment, the invention contemplates the use of such compositions for the formulation and the preparation of biological, pharmaceutical, cosmetic, agricultural, diagnostic or tracing products.

A method for the therapeutic treatment of a medical condition that involves administering, preferably parenterally and more preferably intravenously, to the patient a therapeutically effective dose of the pharmaceutical composition is also within the scope of the invention.

Thus, a method for treating a patient includes administering to the patient a therapeutically effective amount of a compound of the invention.

“Treating” or “treatment” includes the administration of the compositions or compounds of the present invention to a patient who has a disease or disorder (e.g., cancer or metastatic cancer), a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. “Treating” or “treatment” of cancer or metastatic cancer using the methods of the present invention refers to the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with neoplastic disease. The term “therapeutic effect” refers to the reduction, elimination, or prevention of the disease, symptoms of the disease, or side effects of the disease in the subject.

In another aspect of the invention, the compounds of the invention are generally useful for the treatment of many medical conditions including cancer, neoplastic diseases, tumors, inflammatory diseases, and infectious diseases. Examples of preferred diseases for treatment are breast cancer, colorectal cancer, liver cancer, lung cancer, prostate cancer, ovarian cancer, brain cancer, and pancreatic cancer. More specifically, since several specific tumor types have been identified as being positive for CD10, treatment for these types of tumors is especially advantageous with the compounds taught herein. Specifically, treatment for one of the following tumor types may be effected: B-cell lymphoblastic leukemia, T-cell lymphoblastic leukemia, lymphoma, including Hodgkin's lymphoma and non-Hodgkin's lymphoma, follicular lymphoma, Burkitt lymphoma, melanoma, ocular melanoma, cutaneous melanoma, colon adenocarcinomas, hepatocellular carcinomas, renal cell carcinoma, ovarian carcinoma, prostate adenocarcinoma, liver carcinoma, transitional cell carcinoma, pancreatic adenocarcinoma, lung carcinoma, breast carcinoma, and colon carcinoma.

The invention also pertains to uses of the compounds of the invention for the manufacture of a medicament for treating or preventing a disorder selected from the group comprising cellular proliferative and/or differentiative disorders, disorders associated with bone metabolism, immune disorders, hematopoietic disorders, cardiovascular disorders, liver disorders, kidney disorders, muscular disorders, neurological disorders, hematological disorders, viral diseases, pain or metabolic disorders, preferably for treating cancers.

In a preferred embodiment, the invention encompasses pharmaceutical formulations that comprise, or alternatively consist of, at least one compound according to this invention that can be combined with a pharmaceutically acceptable vehicle, vector, diluent or excipient. The terms “pharmaceutically acceptable”, as they refer to vehicle, vector, diluent or excipient, are used interchangeably and represent that the materials are capable of administration to or upon a human without the production of undesirable physiological effects to a degree that would prohibit administration of the composition.

A subject can be treated with a pharmaceutically effective amount of a compound according to the invention. In a preferred embodiment of the invention, the subject is a human subject. The expression “pharmaceutically effective amount” or “effective amount of” means an amount that can induce the biological or medical response of a tissue, system, animal or human as expected by the research worker or the doctor in attendance.

The compositions defined above can also comprise, or be combined with, at least one other medicinal active ingredient or at least one adjuvant that is well known to one skilled in the art, such as for example vitamins, anti-oxidizing agents, to be used in conjunction with the compound according to the invention to improve and to extend the treatment.

The compositions of the invention are particularly useful in that they have a very low toxicity or are not toxic.

The pharmaceutical formulations according to the invention are able to be used in vivo for preventive or curative purposes for diseases or disorders. Non-limiting examples of diseases or disorders for which the pharmaceutical formulations according to the invention may be used include viral infections, cancers, metastases, cellular apoptosis disorders, degenerative diseases, tissue ischemia, infectious diseases or a viral, bacterial or fungal nature, inflammation disorders and pathological neo-angiogenesis.

The administration of the compounds according to the invention can be done by any of the administration methods accepted for the therapeutic agents and generally known in the art. These processes include, but are not limited to, the systemic administration, for example by oral, nasal, parenteral or topical administration, for example by transdermal means or else by central administration, for example by an intracranial surgical path, or else by intraocular administration.

The oral administration can be done by means of tablets, capsules, soft capsules (including formulations with delayed release or extended release), pills, powders, granules, elixirs, dyes, suspensions, syrups and emulsions. This form of presentation is more particularly suited for the passage of the intestinal barrier.

The parenteral administration is done generally by subcutaneous, intramuscular or intravenous injection, or by perfusion. The injectable compositions can be prepared in standard forms, either in suspension or liquid solution or in solid form that is suitable for an extemporaneous dissolution in a liquid.

A possibility for parenteral administration uses the installation of a system with slow release or extended release that ensures the maintenance of a constant dose level. For intranasal administration, it is possible to use suitable intranasal vehicles that are well known to those skilled in the art.

For transdermal administration, it is possible to use transdermal cutaneous patches that are well known to one skilled in the art. A transdermal release system allows for continuous administration. Other preferred topical preparations include, but are not limited to, creams, medicated ointments, lotions, aerosol sprays and gels.

Based on the administration method provided, the compounds of the invention can be in solid, semi-solid or liquid form.

For solid compositions, such as tablets, pills, powders or granules in the free state or included in capsules, the active ingredient can be combined with excipients, such as: a) diluents, for example lactose, dextrose, sucrose, mannitol, sorbitol, cellulose and/or glycine; b) lubricants, for example silica, talc, stearic acid, its magnesium or calcium salt and/or polyethylene glycol; c) binders, for example magnesium silicate and aluminum silicate, starch paste, gelatin, tragacanth gum, methyl cellulose, carboxymethyl cellulose that contains soda and/or polyvinyl pyrrolidone; if necessary, d) disintegrating agents, for example starch, agar, alginic acid or its sodium salt, or effervescent mixtures; and/or e) absorbents, coloring agents, aromatizing agents and sweetening agents.

For semi-solid compositions, such as suppositories, the excipient can be, for example, a fatty emulsion or suspension or can be based on polyalkylene glycol, such as polypropylene glycol.

The liquid compositions, in particular those intended for injection or to be included in a soft capsule, can be prepared by, for example, dissolution, dispersion, etc., of the compound according to the invention in a pharmaceutically pure solvent such as, for example, water, a saline solution of sodium chloride (NaCl), the physiological serum, aqueous dextrose, glycerol, ethanol, an oil and the like.

The compounds according to the invention can also be administered in the form of systems for release of the liposome or lipoplex type, such as in the form of small unilamellar vesicles, large unilamellar vesicles and multilamellar vesicles. The liposomes can be formed from a variety of phospholipids, containing cholesterol, stearylamine or phosphatidylcholines.

The compositions according to the invention can be sterilized and/or can contain one or more of: non-toxic adjuvants and auxiliary substances such as agents for preservation, stabilization, wetting or emulsification; agents that promote dissolution; and salts to regulate osmotic pressure and/or buffers. In addition, they can also contain other substances that offer a therapeutic advantage. The compositions are prepared, respectively, by standard processes of mixing, granulation or coating well known to those skilled in the art.

The dosage for the administration of compounds according to the invention is selected according to a variety of factors including the type, strain, age, weight, sex and medical condition of the subject; the severity of the condition to be treated; the method of administration; the condition of the renal and hepatic functions of the subject and the nature of the particular compound or salt that is used. A normally experienced doctor or veterinarian will easily determine and prescribe the effective amount of the desired compound to prevent, disrupt or stop the progress of the medical condition that is to be treated.

Any of the pharmaceutical compositions above can contain from about 0.1 to about 99%, preferably from about 1 to about 70%, of active ingredient.

By way of examples, when given parenterally, the effective levels of the compounds according to the invention will be in the range of from about 0.002 mg to about 500 mg per kg of body weight and per day.

The compounds according to the invention can be administered in the form of single daily doses, or the total daily dosage can be administered in two, three, four or more doses per day.

In a particular embodiment of the invention is provided a diagnostic agent, for use in vitro, comprising or alternatively consisting of at least one compound according to this invention. The compound according to the embodiment will then have a marker. Such a diagnostic agent can also be used in vivo.

This invention also contemplates in another embodiment a diagnostic kit that comprises said diagnostic agent. More particularly, the diagnostic kit comprises, in one or more containers, a predetermined amount of a composition according to the invention.

Further advantages and characteristics of the invention will emerge from the following examples, given by way of illustration and which are not intended as limiting, and in which reference will be made to the accompanying drawings.

It will be clear that the invention may be practiced otherwise than as particularly described in the foregoing examples. Numerous modifications and variations of the present invention are possible in light of the above teachings and, therefore, are within the scope of the appended claims.

### EXAMPLES

### Example 1

### Characterization of Non-Internalizing Antibodies

1.1. ³H Labelling of Antibodies

Antibodies are labeled with NaB³H₄ by reductive methylation of lysines (Means and Feeney, Biochemistry, 7:2192-2201, 1968). Typically, a specific radioactivity of about 500 dpm/ng of antibodies is obtained.

1.2. Cell Binding of Antibodies

Cells are grown to confluency in 25 cm² flasks and then incubated in 2 ml of culture medium with excess of ³H-labeled antibodies (at 4° C. or 37° C.), for 48 hours (maximum time). After incubation, the culture medium is removed and the cells are rinsed twice with 2 ml of culture medium and three times with phosphate buffered saline (PBS) either at 4° C. or at room temperature. The cells are then lysed in 1 ml of 1% (w/v) sodium deoxycholate adjusted to pH 11.3 followed by a disruption by sonication, and assayed for protein (with serum albumin as standard) and for associated radioactivity after dispersion of the samples in an appropriate cocktail (eg: Aqualuma, Lumac LSC, Groningen, the Netherlands) in a scintillation counter with automatic correction for sample quenching.

1.3. Intracellular Digestion of Antibodies

For experiments at 37° C. (performed as described in 1.2) the culture media are further analyzed for intracellular antibody digestion by measuring the amount of ³H label soluble in 15% (w/v) trichloroacetic acid after precipitation and centrifugation of the culture media proteins. As a control, culture medium containing 3H-labeled antibodies are incubated in parallel at 37° C. but in absence of cells.

1.4. Cell Capture of Antibodies

The amount of antibodies captured by the cells is obtained by the sum of the cell associated antibodies and the amount digested by the cells.

1.5. Microscopic Examination the Cellular Processing of the Antibodies

Antigen bearing cells are cultured for three days on coverslips in cell culture dishes at 4° C. or 37° C. with culture medium containing the antibodies. After incubation, cells are rinsed twice with culture medium and washed three times with PBS and then fixed with 4% (v/v) formaldehyde in PBS for five minutes at room temperature. To permeabilize membranes, cells are incubated for four minutes at room temperature in presence of 0.1% (v/v) Triton X-100 in PBS and washed three times with PBS. Permeabilized cells are then incubated for three hours at room temperature in the presence of peroxidase-conjugated specific “anti-antibody” antibodies. Diaminobenzidine and H₂O₂ are used as substrates for peroxidase (Graham and Karnovsky, J. Histochem. Cytochem., 14: 291-302, 1966). The coverslips are counterstained with hematoxilin, dehydrated and mounted before examination with an optical microscope.

1.6. FACS Analysis of Antibodies Non-Internalization

Cells are incubated in 2 ml PBS containing the antibodies for one hour at 37° C. Cells are then washed three times with cold (4° C.) PBS and incubated in the culture medium at 37° C. after 0, 4, and 18 hours samples of the 10⁵ cells are transferred to an ice bath, washed twice with PBS and incubated with fluorescein isothiocyanate (FITC) conjugated “anti-antibody” antibodies for one hour at 4° C. After washing twice with cold PBS the surface fluorescence of 10⁴ cells is measured with a FACS flow cytometer. A non-internalization of the antibodies is indicated by the stable surface labeling of the cells after 18 hours (maximum time) of incubation at 37° C.

1.7. pH dependence of the cellular capture of the antibodies

To estimate the dissociation of ³H-labeled antibodies bound cells as a function of pH, confluent cultures in 25 cm² flasks are incubated for one hour at 4° C. or 37° C. in the presence of ³H-labeled antibodies. Cells are then rinsed at 4° C. once with cold culture medium and washed three times with cold PBS, and then incubated for ten minutes at 4° C. with 10 mM citrate buffers at different pH values (2; 3; 4; 4.5; 5; 5.5; 6 and 7) in 0.15 M NaCl. After washings with PBS, cell-associated radioactivity and proteins are assayed as described above (see § 1.2).

### Example 2

### Synthesis of a Non-Internalizing Antibody-Ala-Leu-Ala-Leu-Doxorubicine Compound

2.1 Synthesis of Ala-Leu-Ala-Leu-doxorubicine

A solution of doxorubicin.HCl (Meiji, Japan) (400 mg, 0.69 mmol), Fmoc-Ala-Leu-Ala-Leu-OH (500 mg, 0.83 mmol) and diisopropylethylamine (DIPEA) (381 μl, 3.10 mmol) in 5 ml of dimethylformamide (DMF) was stirred for 10 min. To this reaction mixture was added drop wise (340 mg, 0.89 mmol) O-(7-Azabenzotriazol-1-yl)-N,N,N′,N′-tetramethyluronium hexafluorophosphate (HATU) in 2 ml of DMF. The mixture was stirred for 2 hours at room temperature (RT).

The crude was added slowly (over a minimum of 5 minutes) to methyl tert-butyl ether (MTBE) (70 ml) at 0° C. The red precipitate was then filtrated and dried under vacuum (200 mBar) overnight.

To a solution of Fmoc-Ala-Leu-Ala-Leu-doxorubicine (578 mg, 0.5 mmol) in 25 ml of DMF was added 2.5 ml (50 molar equivalents) of Piperidine at 10% in DMF. The reaction was stirred at room temperature for 5 min. To this solution was added slowly 32.5 ml of sodium lactate aqueous 10% (w/v) solution under pH control (pH=3) and at 4° C. The crude was purified using C18 ODS-A YMC gel and reverse-phase preparative HPLC before freeze-drying.

2.2 Synthesis of Non-Internalizing antibody-Ala-Leu-Ala-Leu-doxorubicine

The non-internalizing antibody (50 mg/ml in H₂O) was succinylated using 4 molar equivalents of anhydride succinic acid for 1 molar equivalent of lysine residues in the protein. Anhydride succinic acid was added drop wise and the pH was adjusted to 7.5 by adding NaOH 1N.

After 1 hour at room temperature, the succinylated non-internalizing antibody was dialysed against 0.1M sodium phosphate buffer 0.5 M NaCl at 4° C. for 24 hours (the buffer was changed 4 times) to remove excess of anhydride succinic acid.

The succinylated non-internalizing antibody (350 mg) was diluted to 2 mg/ml in 0.1M sodium phosphate buffer, 0.5M NaCl and 80 molar equivalents of Ala-Leu-Ala-Leu-doxorubicine diluted in 1 ml H₂O are added. 300 molar equivalents of ECDI (ethyl-3-(3-dimethylaminopropyl) carbodiimide chlorhydrate, SIGMA) was added in two steps as an activating agent. The reaction took place at 4° C., for 24 hours and under pH control (pH=8).

The product of the coupling reaction was purified using Sep-pack devices.

The excess of Ala-Leu-Ala-Leu-doxorubicine was removed in a charcoal mixture at room temperature for 30 minutes. Conjugate was filtered through a 0.22 μm device and stored at 4° C.

1. A compound comprising:
(1) a therapeutic agent or marker,
(2) an oligopeptide that can be cleaved selectively by at least one enzyme that is present only or preferably close to or at said target cells, and
(3) a non-internalizing antibody
wherein the non-internalizing antibody hinders cleavage of the compound by enzymes present in whole blood.

2. The compound of claim 1, wherein the oligopeptide is cleaved by at least one enzyme that is present in the environment of one or more tumor cells, stromal cells of tumors, neoangiogenic endothelial cells of tumors and tumor metastases, macrophages, monocytes, polymorphonuclear leukocytes or lymphocytes that infiltrate tumors and tumor metastases.

3. The compound of claim 1, wherein the enzyme is a peptidase, wherein said peptidase is neprilysin (CD10), thimet oligopeptidase (TOP), prostate specific antigen (PSA), plasmin, legumain, collagenase, urokinase, cathepsin, or a matrix metallopeptidase.

4. The compound of claim 1 wherein the oligopeptide comprises one or more of the following amino acid pairings: Arg-Leu, Arg-Phe, Arg-Val, Ala-Phe, Ala-Leu, Ala-Tyr, Cys-Arg, Cys-Asp, Cys-Phe, Gln-Phe, Gly-Asp, Gly-Phe, Gly-Leu, Gly-Gln, Gly-Gly, Gly-Pro, His-Ser, Ile-Ala, Leu-Gln, Leu-Gly, Leu-Leu, Leu-Phe, Leu-Tyr, Lys-Leu, Met-Leu, Pro-Phe, Pro-Tyr, Pro-Leu, Phe-Leu, Phe-Phe, Tyr-Ile, Tyr-Pro, Tyr-Leu, Val-Tyr, Val-Phe, Ser-Leu, and Ser-Lys.

5. The compound of claim 4, wherein the oligopeptide comprises one or more of the following sequences: (Leu)y-(Ala-Leu)x-Ala-Leu or (Leu)y-(Ala-Leu)x-Ala-Phe, wherein Leu is leucine, Ala is alanine, Phe is phenylalanine, y=0 or 1 and x=1, 2, or 3.

6. The compound of claim 1, wherein the oligopeptide comprises one or more of the following sequences: Ala-Phe-Lys (SEQ ID No. 1), Ala-Leu-Ala-Leu (SEQ ID No. 2) or beta-Ala-Leu-Ala-Leu (SEQ ID No. 3), Ala-Leu-Lys-Leu-Leu (SEQ ID No. 4), Ala-Tyr-Gly-Gly-Phe-Leu (SEQ ID No. 5), His-Ser-Ser-Lys-Leu-Gln-Leu (SEQ ID No. 6), Gly-Pro-Leu-Gly-Ee-Ala-Gly-Gln (SEQ ID No. 7), Cys-Asn-Cys-Arg-Gly-Asn-Cys-Phe-Cys (SEQ ID No. 8), Cys-Asp-Cys-Arg-Gly-Asp-Cys-Phe-Cys (SEQ ID No. 9).

7. The compound of claim 1, wherein the therapeutic agent is a chemical agent, a polypeptide, a protein, a nucleic acid, an antibiotic, or a virus.

8. The compound of claim 7, wherein the therapeutic agent has anti-tumor therapeutic activity, anti-angiogenic activity, or anti-inflammatory activity.

9. The compound of claim 7 wherein the therapeutic agent is anthracycline, doxorubicin, daunorubicin, folic acid derivative, vinca alkaloid, calicheamicin, mitoxantrone, cytosine arabinoside, adenosine arabinoside, fludarabine phosphate, melphalan, bleomycin, mitomycin, L-canavanine, taxoid, camptothecin, 9-dimethylaminomethyl-hydroxy-camptothecin hydrochloride, proteasome inhibitor, farnesyl-transferase inhibitors (FTI), epothilone, maytansinoid, discodermolide, fostriecin, platinum derivative, duocarmycin, combretastatin, epipodophyllotoxin, tumor necrosis factor-alpha (TNF-alpha), interferon alpha (IFN-alpha), interferon gamma (IFN-gamma), interleukin 1 (IL-1), IL-2, IL-4, IL-6, IL-10, IL-12, IL-15, or IGF-1 antagonist.

10. (canceled)

11. (canceled)

12. A pharmaceutical composition comprising the compound of claim 1.

13. (canceled)

14. A method for the treatment of cancers or infectious diseases comprising administering an effective amount of the compound of claim 1 to a patient in need thereof.

15. The compound of claim 1, wherein the oligopeptide is indirectly linked to the therapeutic agent through a linker group.

16. The compound of claim 1, wherein the non-internalizing antibody is indirectly linked to the oligopeptide through a spacer group that separates the non-internalizing antibody from the oligopeptide so as to make possible or to facilitate the cleavage of the oligopeptide.

17. The compound of claim 15, wherein the non-internalizing antibody is indirectly linked to the oligopeptide through a spacer group that separates the non-internalizing antibody from the oligopeptide so as to make possible or to facilitate the cleavage of the oligopeptide.

18. The compound of claim 16, wherein the oligopeptide is indirectly linked to the therapeutic agent through a linker group.

19. The compound of claim 16, wherein the spacer group comprises at least one amino acid.

20. The compound of claim 17, wherein said amino acid is in D conformation.

21. The compound of claim 18, wherein said amino acid is a serine in D conformation.


SEQUENCE LISTING (9 sequences)

SEQ ID NO: 1 - protein, 3 aa, Artificial Sequence
  Chemically Synthesized
AFK

SEQ ID NO: 2 - protein, 4 aa, Artificial Sequence
  Chemically Synthesized
ALAL

SEQ ID NO: 3 - protein, 4 aa, Artificial Sequence
  Chemically Synthesized
XLAL

SEQ ID NO: 4 - protein, 5 aa, Artificial Sequence
  Chemically Synthesized
ALKLL

SEQ ID NO: 5 - protein, 6 aa, Artificial Sequence
  Chemically Synthesized
AYGGFL

SEQ ID NO: 6 - protein, 7 aa, Artificial Sequence
  Chemically Synthesized
HSSKLQL

SEQ ID NO: 7 - protein, 8 aa, Artificial Sequence
  Chemically Synthesized
GPLGIAGQ

SEQ ID NO: 8 - protein, 9 aa, Artificial Sequence
  Chemically Synthesized
CNCRGNCFC

SEQ ID NO: 9 - protein, 9 aa, Artificial Sequence
  Chemically Synthesized
CDCRGDCFC